(19) United States
(12) Patent Application Publication
(10) Pub. No.: US 2011/0245916 A1
(43) Pub. Date: Oct. 6, 2011

(54) POLYMER VALVE AND PULSATILE CONDUIT-TYPE VAD USING THE SAME

(76) Inventors: Byoung Goo MIN, Goyang-si (KR); Jung Chan LEE, Seoul (KR); Yong Soon WON, Seoul (KR); Wha Ryong KIM, Seoul (KR); Pil Kyeong SHIN, Seoul (KR)

(21) Appl. No.: 12/964,180

(22) Filed: Dec. 9, 2010

(30) Foreign Application Priority Data
Apr. 6, 2010 (KR) 10-2010-0031234

(51) Int. Cl. A61F 2/24; A61M 1/10

(52) U.S. Cl. 623/2.2; 623/3.1

(57) ABSTRACT

A prosthetic blood valve comprises a base body having a hollow, opposing props extending from the body to partition opposing inclined ridges of the body to allow the inclined ridges and each top of the props to define the hollow and to form an upper surface of the body, and a singular leaflet attached to and along the inclined ridges and the prop tops to cover the hollow. The singular leaflet has a substantially central opening between the props and formed of a flexible material so a forward or outward blood flow passes through the opening and a reverse flow or backflow of blood leads to opposing leaflet portions around the opening being abuttingly pulled to each other to thereby prevent the blood backflow.

### CROSS REFERENCE

This application claims foreign priority under Paris Convention and 35 U.S.C. §119 to Korean Patent Application No. 10-2010-0031234, filed Apr. 6, 2010 with the Korean Intellectual Property Office.

### BACKGROUND OF THE INVENTION

This invention relates to a ventricular assist device (VAD), and more particularly to an improved polymer valve and a pulsatile conduit-type ventricular assist device using the polymer valve to substantially expand a device life span and durability.

Heart valves maintain the unidirectional flow of blood in the heart by opening and closing depending on the difference in pressure on each side. A human heart carries four valves: a bicuspid valve between a right atrium and a right ventricle; a pulmonary valve between a pulmonary artery and a right ventricle; an aortic valve between a aorta and a left ventricle; and a mitral valve between a left atrium and a left ventricle. In recent years, artificial heart valves have been developed to replace malfunctioning heart valves. Artificial heart valves include mechanical, biological and polymer valves.

One of the major drawbacks of mechanical heart valves is that patients with these implants require consistent anti-coagulation therapy. Clots formed by red blood cell and platelet damage can block up blood vessels and lead to very serious consequences. Clotting occurs in one of three basic pathways: tissue factor exposure, platelet activation, or contact activation by foreign materials, and in three steps: initiation, amplification, and propagation.

In order to overcome the limitation of such mechanical heart valves, biological valves are introduced in 1969. Biological valves are valves of animals, like pigs, which undergo several chemical procedures in order to make them suitable for implantation in the human heart. There are some risks associated with a biological valve such as the human body's tendency to reject foreign material, thus resulting in a replacement implantation in about ten to fifteen years.

A recent introduction is a polymer valve which is cost effective compared to mechanical or biological valve and enables fabrication of a desired shape with ease. A polymer valve varies to a bi-leaflet and tri-leaflet type.

U.S. Pat. No. 6,579,223 discloses an artificial valve having a stretchable bladder with a blood inlet and a blood outlet. for pumping blood to stretch and expand during the blood filling phase, and elastically contract to its normal size during the blood ejection phase.

U.S. Pat. No. 6,958,076 discloses an artificial hear valve comprising opposing pliable nonbiological leaflet members to shift between a first normally open position so blood may flow in a first direction through an axial passageway and a second closed position so blood fluid is prevented from backflowing through the axial passageway. The leaflets are normally open and only closing upon reversal of fluid flow to the second direction, and an elongated hollow support member receives the leaflet members.

Such conventional device has limitation since a metallic material is employed in the valve which may result in generation of unwanted thrombus in the blood system and limitation in dispersing backflow pressure, without which a valve needs to be replaced in a frequent manner. The valve replacement means an additional cost to a patient and lack of valve durability.

### SUMMARY OF THE INVENTION

The invention is contrived to overcome the conventional disadvantages. Accordingly, an objective of the present invention is to provide a polymer valve to evenly distribute force and pressure applied to the leaflets to improve a valve life span and durability.

Another objective is to provide a polymer valve for a VAD with a cost-effective simplified structure to save more patients suffering from blood valve malfunctioning.

A further objection is to provide an improved polymer valve and a pulsatile conduit-type VAD that substantially prevents blood clotting (thrombosis) and dissolution or destruction of red blood cells from occurring in blood vessels of a heart patient who receives its assistance by employing non-metallic bio-friendly materials.

A still further objective of the prevent invention is to enable a heart patient to use the blood valve for a longer time period in form of either extracorporeal life support or surgical implantation.

To achieve these and other objectives, a prosthetic blood valve comprises a base body having a hollow therethrough; opposing props extending from the body to partition opposing inclined ridges of the body so the inclined ridges and each top of the props define the hollow and form an upper surface of the body; and a singular leaflet attached to and along the inclined ridges and the prop tops to cover the hollow, wherein the singular leaflet has a substantially central opening between the props so the leaflet is formed of a flexible material, whereby a forward or outward blood flow passes through the opening and a reverse flow or backflow of blood leads to opposing leaflet portions around the opening being abuttingly pulled to each other to thereby prevent the blood backflow.

In the prosthetic blood valve, the singular leaflet may be formed of a polymer film, preferably a polyurethane film. The opening is substantially shaped in a letter ‘U’ when viewed from a side of the inclined ridges. The U-shaped opening serves to evenly distribute a blood flow pressure and force toward the singular leaflet. The opening is substantially shaped in a top-down arch when viewed from a side of the inclined ridges. Here, the arch-shaped opening serves to evenly distribute a blood flow pressure and force toward the singular leaflet.

In an embodiment, a blood pump device comprise first and second prosthetic blood valves aligned to allow a blood flow from the first value to the second valve; an elastic conduit formed between the blood valves; and a housing to enclose the conduit, wherein said each blood valve comprises: a base body having a hollow therethrough; opposing props extending from the body to partition opposing inclined ridges of the body, wherein the inclined ridges and each top of the props define the hollow and form an upper surface of the body; and a singular leaflet attached to and along the inclined ridges and the prop tops to cover the hollow, wherein the singular leaflet has a substantially central opening between the props, wherein the leaflet is formed of a flexible material, whereby a forward or outward blood flow passes through the opening and a reverse flow or backflow of blood leads to opposing leaflet portions around the opening being abuttingly pulled to each other to thereby prevent the blood backflow; whereby an increased air pressure in the housing serves to deflate the conduit which leads to a blood outflow through the second valve opening with the first valve opening closed and a decreased air pressure in the housing serves to inflate the conduit which leads to a blood inflow from the first valve through the first valve opening with the second valve opening closed. The blood pump device may further comprise an air pump connected to the housing to control the air pressure in the housing.

Although the present invention is briefly summarized, the fuller understanding of the invention can be obtained by the following drawings, detailed description and appended

### BRIEF DESCRIPTION OF THE DRAWINGS

These and other features, aspects and advantages of the present invention will become better understood with reference to the accompanying drawings, wherein:

FIG. 1 is a perspective view showing a prosthetic blood valve according to the present invention;

FIG. 2 is a perspective view showing a body part of the prosthetic blood valve according to the present invention;

FIGS. 3A and 3B are perspective views showing preferred embodiments of a prosthetic blood valve according to the present invention;

FIGS. 4A through 4D are views showing molding steps to fabricate the prosthetic blood valve according to the present invention;

FIG. 5 is a schematic prospective view showing a blood pump device or ventricular assist device using prosthetic blood valves according to the present invention; and

FIGS. 6A and 6B are schematic views showing mechanism of blood flow through the blood pump or ventricular assist device according the present invention.

### DETAILED DESCRIPTION OF THE PREFERRED EMBODIMENTS

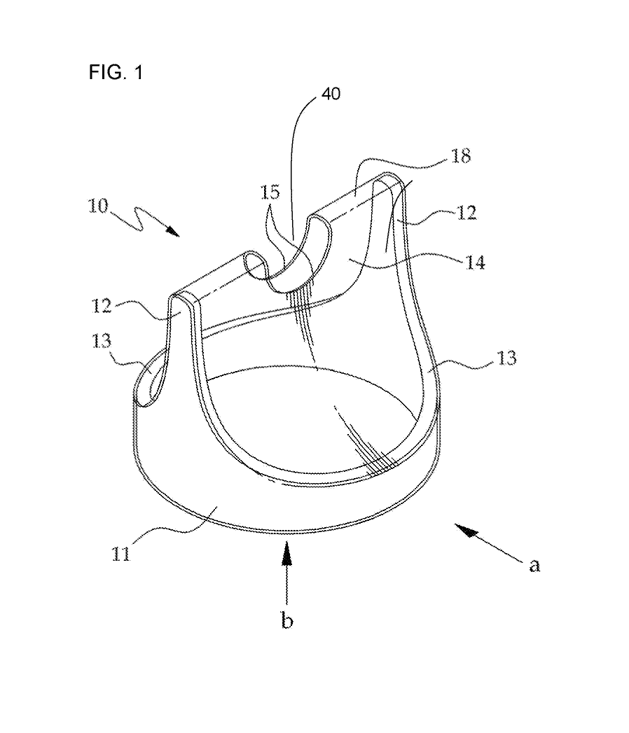

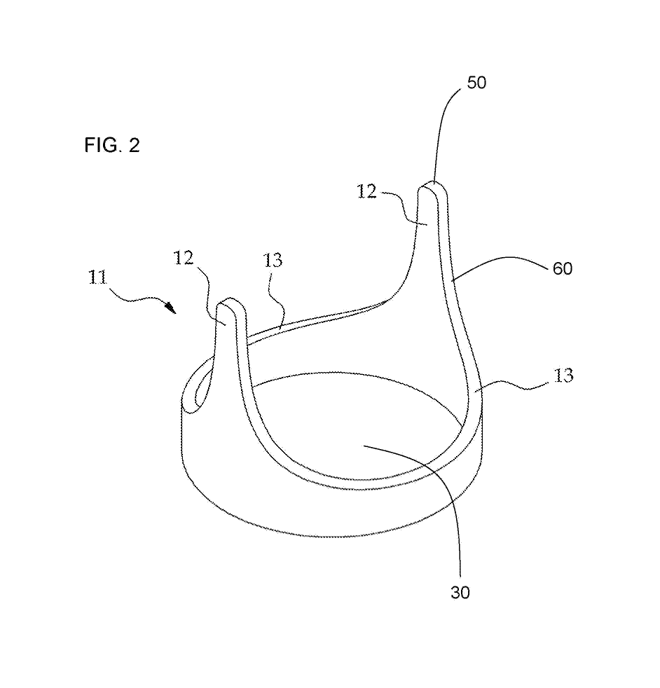

Referring to the accompanying drawings, preferred embodiments of the present invention will now be explained. FIGS. 1 and 2 illustrates construction of a prosthetic blood valve 10. As shown therein, the prosthetic blood valve 10 comprises a base body 11 having a hollow 30 therethrough. Extended from the base body 11 are opposing props 12 to partition opposing inclined ridges 13 of the body 10. Here, the inclined ridges 13 and each top 50 of the props 12 are provided to define the hollow 30 and form an upper surface 60 of the body 11.

For a better performance, the prosthetic blood valve 10 further comprises a singular leaflet 14 attached to and along the inclined ridges 13 and the prop tops 50 to cover the hollow 30. In this construction, the singular leaflet 14 has a substantially central opening 40 between the props 12. It is preferred that the leaflet 14 is formed of a flexible material. This construction results in a refined mechanism in which a forward or outward blood flow passes through the opening 40 and a reverse flow or backflow of blood leads to opposing leaflet portions 15 around the opening 40 being abuttingly pulled to each other to thereby prevent the blood backflow.

In an embodiment, the singular leaflet 14 is formed of a polymer film. The singular leaflet 14 may be formed of a polyurethane film. The opening 40 is substantially shaped in a letter ‘U’ when viewed from a side of the inclined ridges 13 so that the U-shaped opening 40 serves to evenly distribute a blood flow pressure and force toward the singular leaflet 14. Alternately, the opening 40 is substantially shaped in a top-down arch when viewed from a side of the inclined ridges 13 so that the arch-shaped opening serves to evenly distribute a blood flow pressure and force toward the singular leaflet.

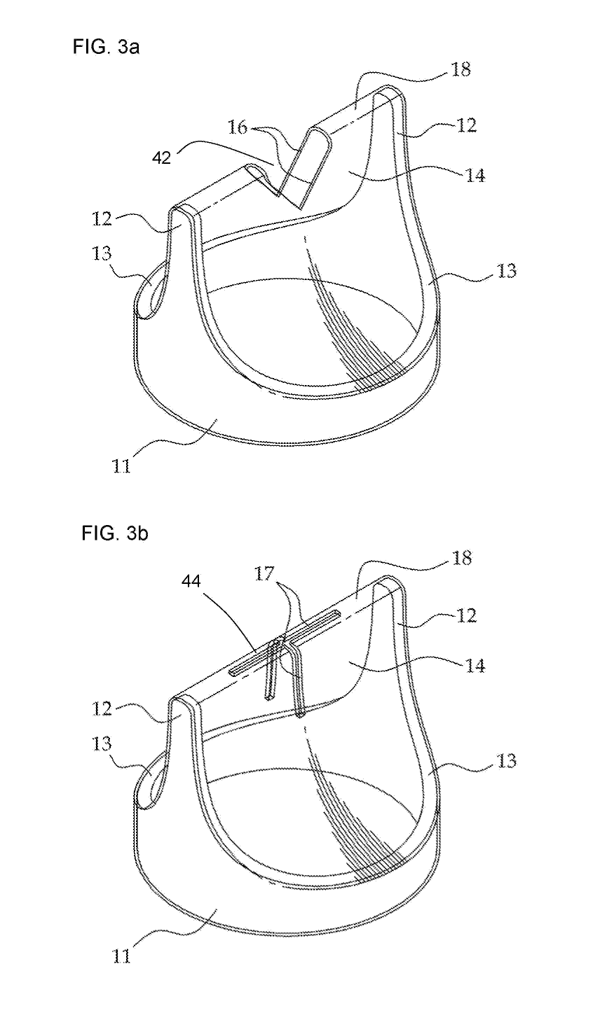

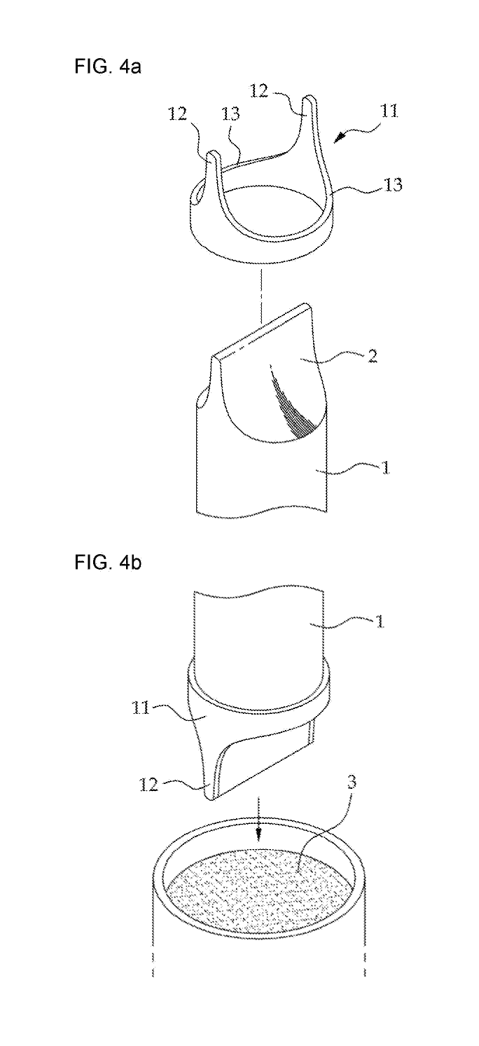

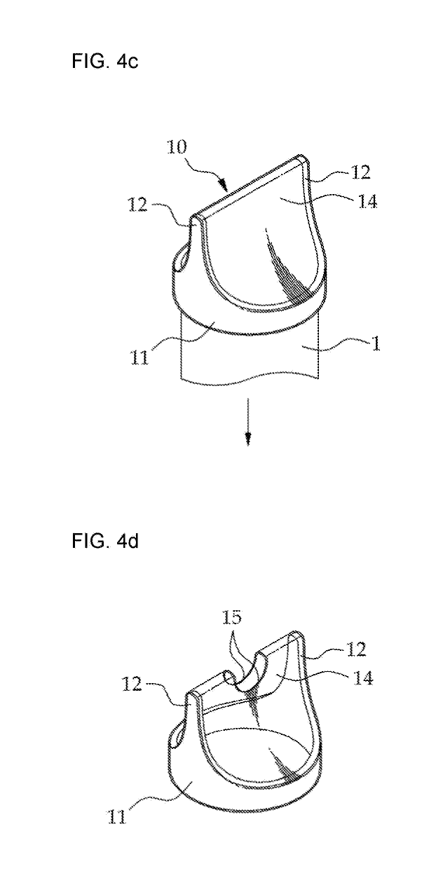

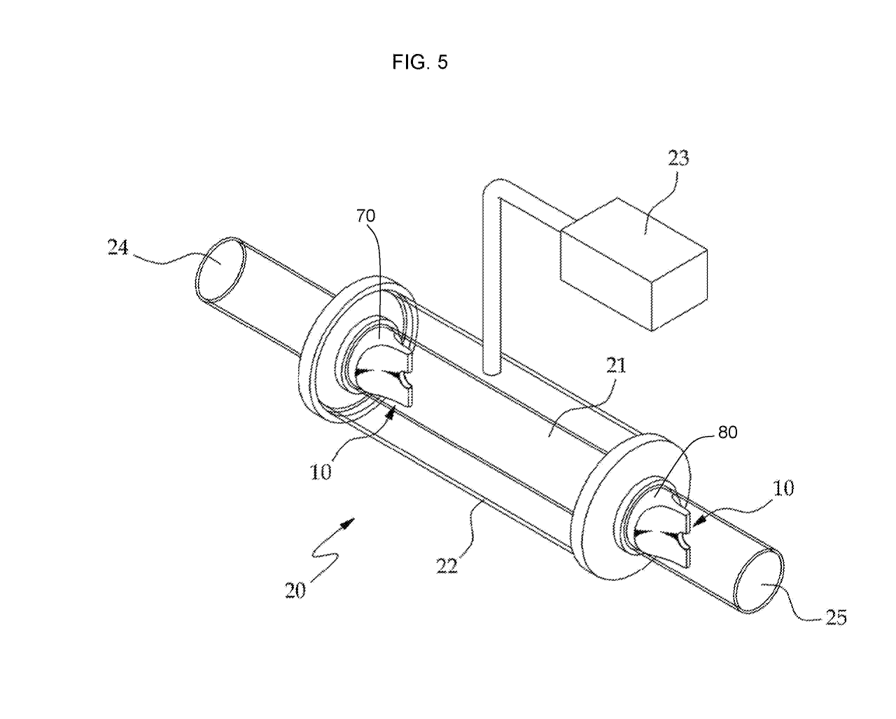

FIGS. 3A and 3B each illustrate variations of the openings 42 and 44, and FIGS. 4A to 4D show molding steps to form the valve 10, which will be further explained below.

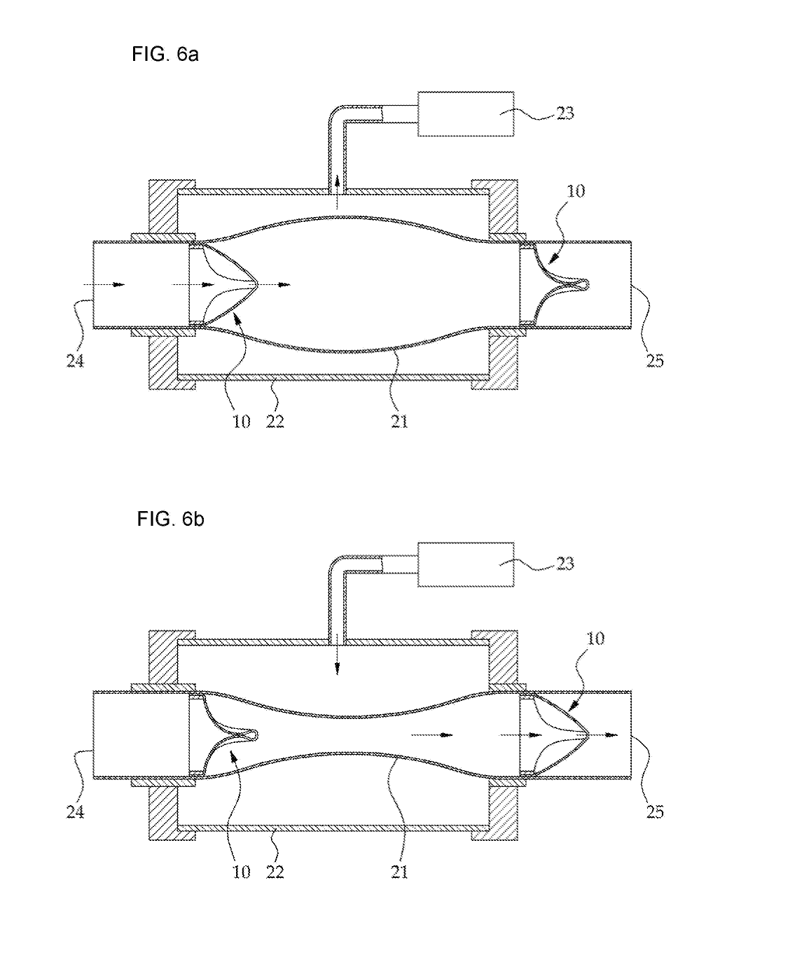

FIGS. 5 and 6A-6B show another embodiment of the present invention to introduce a ventricular assist device or a blood pump device 20. As shown therein, the blood pump device 20 comprises first and second prosthetic blood valves 70 and 80 aligned to allow a blood flow from the first value 70 to the second valve 80. The blood pump device 20 further comprises an elastic conduit 21 formed between the blood valves 70 and 80, and a housing 22 to enclose the conduit 21.

In this construction, each blood valve 70 and 80 comprises, as shown back in FIGS. 1 and 2, the base body 10 having a hollow 30 therethrough; opposing props 12 extending from the body 11 to partition opposing inclined ridges 13 of the body 11 so the inclined ridges 13 and each top 50 of the props 12 define the hollow 30 and form an upper surface 30 of the body 11; and a singular leaflet 14 attached to and along the inclined ridges 13 and the prop tops 50 to cover the hollow 30 so the singular leaflet 14 has a substantially central opening 40 between the props 12 and the leaflet 14 is formed of a flexible material to achieve that a forward or outward blood flow passes through the opening 40 and a reverse flow or backflow of blood leads to opposing leaflet portions 15 around the opening 40 being abuttingly pulled to each other to thereby prevent the blood backflow.

Under this construction, the ventricular assist device or a blood pump device 20 enables that an increased air pressure in the housing 22 serves to deflate the conduit 21 which leads to a blood outflow through the second valve opening with the first valve opening closed and a decreased air pressure in the housing 22 serves to inflate the conduit 21 which leads to a blood inflow from the first valve 70 through the first valve opening with the second valve opening closed.

The ventricular assist device or a blood pump device 20 may further comprise an air pump control 23 connected to the housing 22 to control the air pressure in the housing 22.

As discussed above, the prosthetic blood valve 10 and the blood pump device 20 serve to evenly distribute force and pressure applied to the leaflet to improve a valve life span and durability. The cost-effective simplified structure of the blood valve 10 would lead to saving more patients suffering from heart valve malfunctioning. Further, the blood valve using a polymer leaflet 14 in a singular format serves to prevent blood clotting or thrombosis and dissolution or destruction of red blood cells from occurring in blood vessels of a heart patient who receives its assistance by employing non-metallic bio-friendly materials, thereby enabling a heart patient to live a longer life with support in form of either extracorporeal life support or surgical implantation.

The mechanism of the prosthetic blood valve 10 and the blood pump device 20 will now be further explained with reference to the accompanying drawings. The opposing props 12 are preferably provided in pair. The ridges 13 are preferably formed in a downward slope on each side of the props 12. It is recommended that the prop pair 12 are formed symmetric to each other and consequently the ridges 13 are symmetric to each other perpendicular to the alignment of the props 12. The base body 11 and props 12 serve to support the singular leaflet 14 so be better formed of a bio-friendly material with an appropriate hardness. For example, a bio-friendly polycarbonate or polyurethane can be used for the body 11 and props 12.

The singular leaflet 14 is better formed of a flexible and pliable material. The leaflet 14 covers the hollow 30 of the body 11 and is sealed to and along the ridges 13 and prop tops 50. The leaflet 14 is better formed of a bio-friendly polymer material, preferably, a medical-purpose polyurethane. The leaflet 14 allows a blood inflow through the opening 40 along the arrow b in FIG. 1 and also serves to prevent a backflow of the blood in which the leaflet portions 15 around the opening 50 are pulled to each other thereby closing the opening 50 and blocking the backflow of the blood.

The opening 40 is better formed of a letter ‘U’ when viewed from a side of the ridges 13 or from the arrow a in FIG. 2. The U-shaped opening 40 serves to allow an increased unit flow per hour while spreading or distributing the pressure and force applied to the leaflet 14 during open and close sessions of the opening 40, thereby securing an improved durability and increase life span of the singular leaflet 14.

Selectively, the opening may be formed in a V-shape 42 as shown in FIG. 3A, or in a slit 44 as shown in FIG. 3B. The format of the leaflet opening may vary depending on a target patient and medical record of the patient so as to effectively control the amount of the unit blood flow.

Fabrication steps of the blood valve 10 are illustrated in FIGS. 4A to 4D. As shown therein, a mold 1 is formed and inserted in the base body 11 through the hollow 30. The mold 1 has opposing slant sides 2 corresponding to the leaflet 14 to align with the ridges 13 of the body 11. Then, as shown in FIG. 4B, the mold 1 with the body 11 is dipped in a polymer liquid 3 and taken out for dry using a dip casting method to laminate the mold 1 and the upper surface 60 of the body 11 with the polymer. This dip casting step may be repeated with a predetermined interval to improve quality and durability of the leaflet 14. In FIG. 4C, the mold 1 is detached from the body 11 and the opening 40 is formed by partially cutting out a central portion of the leaflet using a known cutting tool to fabricate the blood valve 10 with the leaflet 14.

Referring to FIGS. 5 and 6A-6B, the ventricular assist device or blood pump device 20 will now be explained in further detail. The entrance 24 serves to communicate with a ventricle of a patient and the exit 25 communicates with an aorta of the patient. When the air inside the case 22 is suctioned out or deflated using the air pump control 23, the conduit 21 comes to inflate or expand since the lowered pressure in the case 22 triggers inflation of the conduit 21. Consequently, the inflation of the conduit 21 causes the blood to flow in through the first blood valve 70 with the second blood valve 80 serving to block backflow. That is, the inflation of the conduit 21 leads the opening of the first valve 70 to open while closing the opening of the second valve 80. Here, the pliable, elastic characteristic of the leaflet of the second valve is closed thereby preventing blood backflow.

As shown in FIG. 6B, when the air pump 23 inflates the case 22 then the increased air pressure inside the case 22 leads to deflate the conduit 21 as much thereby moving the blood inside the conduit 21 through the second blood valve 80 to the exit 25 with the opening of the first blood valve serving to block backflow.

In an embodiment, the conduit 21 serves as an apical aortic conduit (AAC) so that when a ventricle pumps out blood, then the pumped out blood flows into the conduit 21 and the blood moves out through the second blood valve 80 to the exit 25 which communicates with an aorta. This will decrease load to the ventricle thereby curing a heart failure. A highly elastic material for the conduit 21 would maximize an effective decrease of load to the ventricle.

The air pump control 23 employed in the ventricular assist device 20 according to the present invention is formed to serve as AAC so that the malfunctioning of the air pump control 23 would not affect the heart functioning of the patient. Further, when the blood pump device 20 is provided such that it could be implanted, the patient can manually connect the air pump control 23 to the case 23 at home and detach the control 23 for the patient to go out for an outdoor activity, thereby maximizing product reliability,

Although the invention has been described in considerable detail with reference to certain preferred versions thereof, other versions are possible by converting the aforementioned construction. Therefore, the scope of the invention shall not be limited by the specification specified above and the appended claims.

What is claimed is:

1. A prosthetic blood valve, comprising:
a base body having a hollow therethrough;
opposing props extending from the body to partition opposing inclined ridges of the body, wherein the inclined ridges and each top of the props define the hollow and form an upper surface of the body; and
a singular leaflet attached to and along the inclined ridges and the prop tops to cover the hollow, wherein the singular leaflet has a substantially central opening between the props, wherein the leaflet is formed of a flexible material, whereby a forward or outward blood flow passes through the opening and a reverse flow or backflow of blood leads to opposing leaflet portions around the opening being abuttingly pulled to each other to thereby prevent the blood backflow.

2. The prosthetic blood valve of claim 1, wherein the singular leaflet is formed of a polymer film.

3. The prosthetic blood valve of claim 1, wherein the singular leaflet is formed of a polyurethane film.

4. The prosthetic blood valve of claim 1, wherein the opening is substantially shaped in a letter ‘U’ when viewed from a side of the inclined ridges.

5. The prosthetic blood valve of claim 4, wherein the U-shaped opening serves to evenly distribute a blood flow pressure and force toward the singular leaflet.

6. The prosthetic blood valve of claim 1, wherein the opening is substantially shaped in a top-down arch when viewed from a side of the inclined ridges.

7. The prosthetic blood valve of claim 6, wherein the arch-shaped opening serves to evenly distribute a blood flow pressure and force toward the singular leaflet.

8. A blood pump device, comprising:
first and second prosthetic blood valves aligned to allow a blood flow from the first value to the second valve;
an elastic conduit formed between the blood valves; and
a housing to enclose the conduit,
wherein said each blood valve comprises:
a base body having a hollow therethrough;
opposing props extending from the body to partition opposing inclined ridges of the body, wherein the inclined ridges and each top of the props define the hollow and form an upper surface of the body; and
a singular leaflet attached to and along the inclined ridges and the prop tops to cover the hollow, wherein the singular leaflet has a substantially central opening between the props, wherein the leaflet is formed of a flexible material, whereby a forward or outward blood flow passes through the opening and a reverse flow or backflow of blood leads to opposing leaflet portions around the opening being abuttingly pulled to each other to thereby prevent the blood backflow;
whereby an increased air pressure in the housing serves to deflate the conduit which leads to a blood outflow through the second valve opening with the first valve opening closed and a decreased air pressure in the housing serves to inflate the conduit which leads to a blood inflow from the first valve through the first valve opening with the second valve opening closed.

9. The blood pump device of claim 8, further comprising an air pump connected to the housing to control the air pressure in the housing.

10. The prosthetic blood valve of claim 8, wherein the singular leaflet is formed of a polymer film.

11. The prosthetic blood valve of claim 8, wherein the singular leaflet is formed of a polyurethane film.

12. The prosthetic blood valve of claim 8, wherein the opening is substantially shaped in a letter ‘U’ when viewed from a side of the inclined ridges.

13. The prosthetic blood valve of claim 12, wherein the U-shaped opening serves to evenly distribute a blood flow pressure and force toward the singular leaflet.

14. The prosthetic blood valve of claim 8, wherein the opening is substantially shaped in a top-down arch when viewed from a side of the inclined ridges.

15. The prosthetic blood valve of claim 14, wherein the arch-shaped opening serves to evenly distribute a blood flow pressure and force toward the singular leaflet.
